(19) United States
(12) Patent Application Publication
(10) Pub. No.: US 2016/0000401 A1
(43) Pub. Date: Jan. 7, 2016

(54) METHOD AND SYSTEMS FOR ADJUSTING AN IMAGING PROTOCOL

(71) Applicant: GENERAL ELECTRIC COMPANY, Schenectady, NY (US)

(72) Inventor: Martin Mienkina, Neumarkt am Wallersee (AT)

(21) Appl. No.: 14/325,137

(22) Filed: Jul. 7, 2014

(51) Int. Cl. A61B 8/08; A61B 8/00; C12Q 1/68; A61B 8/14

(52) U.S. Cl. CPC A61B 8/0866; A61B 8/14; A61B 8/54; A61B 8/463; C12Q 1/68

(57) ABSTRACT

Various methods and systems are provided for imaging a fetus. One example method for imaging a fetus includes obtaining genetic analysis information of the fetus, and adjusting an ultrasound image acquisition protocol based on the information.

### FIELD

Embodiments of the subject matter disclosed herein relate generally to medical imaging systems, and more particularly to workflows for operating ultrasound imaging systems.

### BACKGROUND

Ultrasound imaging is a noninvasive method for monitoring the growth of a fetus during pregnancy. Images of the fetus acquired with ultrasound may be assessed to determine a relative risk probability that the fetus is affected with one or more developmental disorders. However, such assessments rely on the measurement of one or more soft markers that are correlated with the one or more development disorders, but yet also present in many fetuses not having any development disorders. Thus, the assessments are generally used as a screening tool to identify high-risk patients for further testing. The further testing may include amniocentesis, for example, which poses risks to the fetus.

### BRIEF DESCRIPTION

In one embodiment, a method for imaging a fetus comprises obtaining genetic analysis information of the fetus and adjusting an ultrasound image acquisition protocol based on the information.

Thus, an ultrasound image acquisition protocol may be adjusted based on genetic analysis information, such as a probability of the fetus displaying a particular phenotype (e.g., a developmental disorder such as trisomy 21), as determined by a genetic analysis performed on the fetus prior to the imaging. The genetic analysis may include a sequence analysis of fetal DNA reconstructed from a maternal blood sample. Based on the results of the genetic analysis, the ultrasound imaging protocol may be adjusted to target specific soft markers (e.g., anatomical structures and features) known to be correlated with the disorder. An updated probability of the fetus having the phenotype may be calculated, or a severity of the phenotype may be determined, based on the results of the imaging. In this way, a targeted ultrasound protocol may be followed in light of the genetic analysis, to provide for imaging of specific anatomical structures and/or measurement of particular anatomical features that may not be imaged during a standard ultrasound procedure.

### BRIEF DESCRIPTION OF THE DRAWINGS

The disclosure will be better understood from reading the following description of non-limiting embodiments, with reference to the attached drawings, wherein below:

FIG. 1 schematically shows an example ultrasound system.

FIG. 2 is a flow chart illustrating a method for imaging a fetus according to embodiment of the disclosure.

FIG. 3 is a flow chart illustrating a method for imaging a fetus according to another embodiment of the disclosure.

FIG. 4 schematically illustrates example user interfaces displayed on a display device of an ultrasound system during two exemplary ultrasound image acquisition protocols.

### DETAILED DESCRIPTION

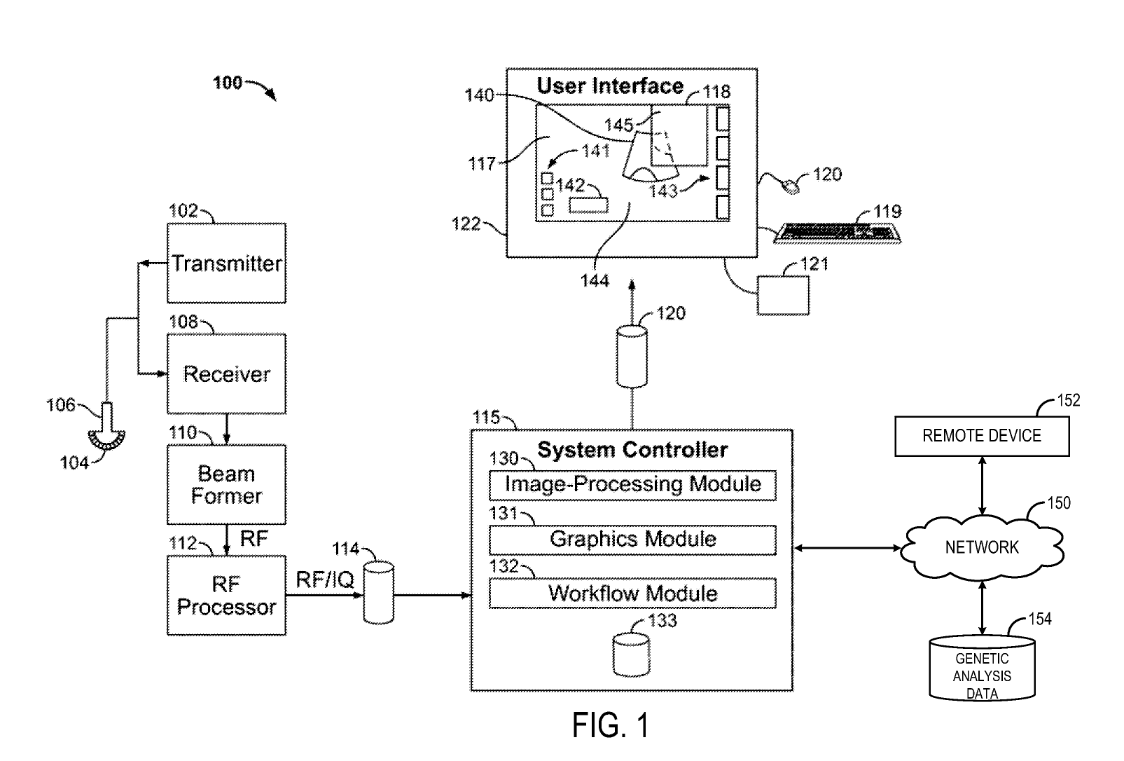

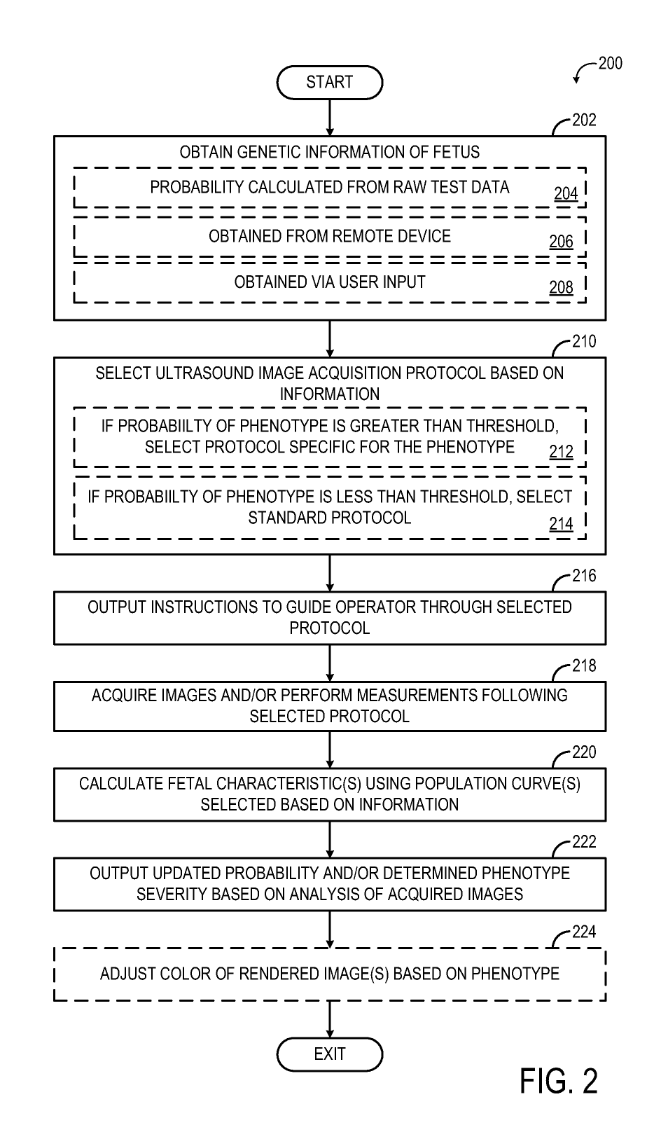

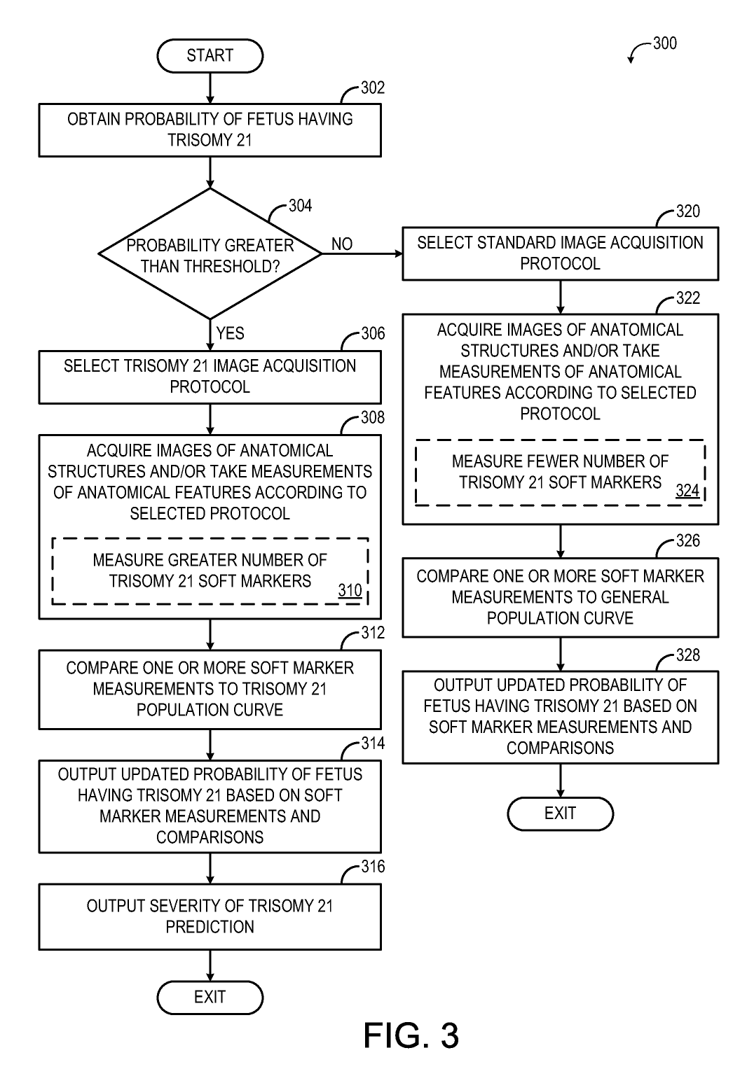

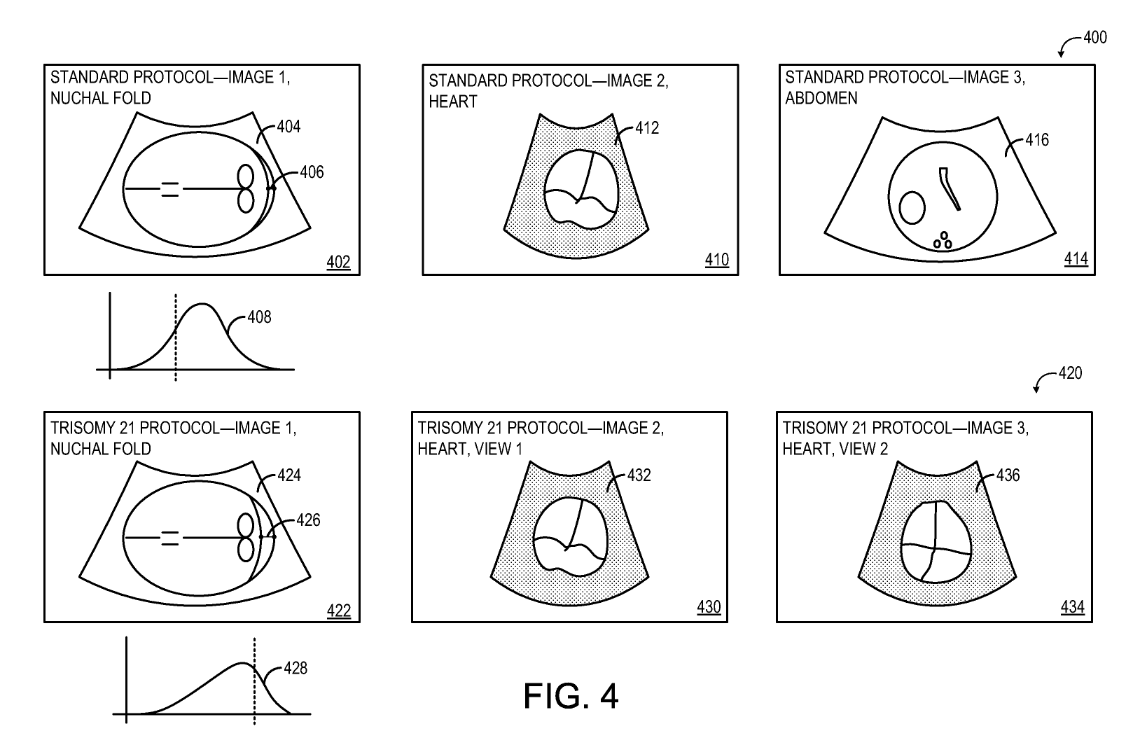

Embodiments of the subject matter disclosed herein relate to a method for imaging a fetus that includes obtaining genetic analysis information of the fetus and adjusting an ultrasound image acquisition protocol based on the information. In one example, the genetic analysis of the fetus may include a DNA sequence analysis performed on fetal DNA reconstructed from a maternal blood sample. This type of fetal DNA analysis may be performed relatively early during pregnancy, and poses little to no risks to the fetus or the mother. However, other types of genetic analysis may be used, such as amniocentesis or parental DNA testing. Based on the results of the genetic analysis, an imaging protocol used to acquire a plurality of images of the fetus may be adjusted so that desired anatomical structures and features may be imaged and measured during the ultrasound, improving diagnostic results and increasing the efficiency of the imaging workflow (e.g., reducing the number of unnecessary images and/or increasing the number of relevant images). While the imaging protocol described herein includes the acquisition of ultrasound images, other medical imaging procedures are possible, such as x-ray, MRI, etc. Further, updating an imaging procedure based on genetic analysis information is herein presented with respect to a fetus, but is not necessarily limited to prenatal imaging and diagnostics. For example, genetic analysis could be performed on a tumor of a patient, and imaging of the tumor may be adjusted based on the genetic analysis of the tumor. An example ultrasound system is presented in FIG. 1, including a computing system having instructions to execute methods for adjusting an ultrasound image acquisition protocol, as illustrated in FIGS. 2 and 3. Example user interfaces displayed during various ultrasound image acquisition protocols are illustrated in FIG. 4.

FIG. 1 illustrates a block diagram of an ultrasound system 100 according to one embodiment. The ultrasound system 100 may be a unitary apparatus such that the elements and components of the system 100 may be carried or moved with each other. However, in other embodiments, at least one of the system components and elements described herein may be located remotely with respect to other components and elements. For example, one or more of the described modules may operate in a data server that has a distinct and remote location with respect to an ultrasound probe and the user interface.

In the illustrated embodiment, the ultrasound system 100 includes a transmitter 102 that drives an array of elements 104, for example, piezoelectric crystals, within a diagnostic ultrasound probe 106 (or transducer) to emit pulsed ultrasonic signals into a body or volume (not shown) of a subject. The elements 104 and the probe 106 may have a variety of geometries. The ultrasonic signals are back-scattered from structures in the body, for example, blood cells or muscular tissue, to produce echoes that return to the elements 104. The echoes are received by a receiver 108. The received echoes are provided to a beamformer 110 that performs beamforming and outputs an RF signal. The RF signal is then provided to an RF processor 112 that processes the RF signal. Alternatively, the RF processor 112 may include a complex demodulator (not shown) that demodulates the RF signal to form IQ data pairs representative of the echo signals. The RF or IQ signal data may then be provided directly to a memory 114 for storage (for example, temporary storage). In some examples, the IQ demodulation may be performed before beamforming, and an IQ-based beamformer may be used. In the illustrated embodiment, the probe 106 is only configured for imaging. In other embodiments, the probe 106 may also be configured to provide therapy through, for example, high-intensity focused ultrasound (HIFU).

The ultrasound system 100 also includes a system controller 115 that includes a plurality of modules. The system controller 115 is configured to control operation of the ultrasound system 100. For example, the system controller 115 may include an image-processing module 130 that receives the ultrasound signals (for example, RF signal data or IQ data pairs) and processes the ultrasound signals to generate frames of ultrasound information (e.g., ultrasound images) for displaying to an operator. The image-processing module 130 may be configured to perform one or more processing operations according to a plurality of selectable ultrasound modalities on the acquired ultrasound information. By way of example only, the ultrasound modalities may include color-flow, acoustic radiation force imaging (ARFI), B-mode, A-mode, M-mode, spectral Doppler, acoustic streaming, tissue Doppler module, C-scan, and elastography. The generated ultrasound images may be two-dimensional (2D) or three-dimensional (3D).

Acquired ultrasound information may be processed in real-time during an imaging session (or scanning session) as the echo signals are received. Additionally or alternatively, the ultrasound information may be stored temporarily in the memory 114 during an imaging session and processed in less than real-time in a live or off-line operation. An image memory 120 is included for storing processed frames of acquired ultrasound information that are not scheduled to be displayed immediately. The image memory 120 may comprise any known data storage medium, for example, a permanent storage medium, removable storage medium, and the like.

In operation, the ultrasound system 100 acquires data, for example, volumetric data sets by various techniques (for example, 3D scanning, real-time 3D imaging, volume scanning, 2D scanning with transducers having positioning sensors, freehand scanning using a voxel correlation technique, scanning using 2D or matrix array transducers, and the like). Ultrasound images are displayed to the operator or user of the ultrasound system 100 on the display device 118.

The system controller 115 is operably connected to a user interface 122 that enables an operator to control at least some of the operations of the image-processing module 130. The user interface 122 may include hardware, firmware, software, or a combination thereof that enables an individual (e.g., an operator) to directly or indirectly control operation of the ultrasound system 100 and the various components thereof. As shown, the user interface 122 includes a display device 118 having a display area 117. In some embodiments, the user interface 122 may also include one or more input devices, such as a physical keyboard 119, mouse 120, and/or touchpad 121. In an exemplary embodiment, the display device 118 is a touch-sensitive display (e.g., touchscreen) that can detect a presence of a touch from the operator on the display area 117 and can also identify a location of the touch in the display area 117. The touch may be applied by, for example, at least one of an individual's hand, glove, stylus, or the like. As such, the touch-sensitive display may also be characterized as an input device that is configured to receive inputs from the operator. The display device 118 also communicates information to the operator by displaying the information to the operator. The display device 118 and/or the user interface 122 may also communicative audibly. The display device 118 is configured to present information to the operator during the imaging session. The information presented may include ultrasound images, graphical elements, user-selectable elements, and other information (e.g., administrative information, personal information of the patient, and the like). Further, in some examples, the information presented may include instructions to guide the operator through a selected ultrasound image acquisition protocol via the workflow module 132, described below.

The system controller 115 also includes a graphics module 131, a workflow module 132, and a database 133. The image-processing module 130, the graphics module 131, and the workflow module 132 coordinate with one another to present information to the operator during the imaging session. For example, the image-processing module 130 may be configured to display an acquired image 140 on the display device 118, and the graphics module 131 may be configured to display designated graphics along with the ultrasound images. The graphics may include icons 141, data fields 142, user-selectable elements 143, and the like.

The workflow module 132 may be configured to guide and assist the operator during the imaging session by showing various screen configurations for different stages of the imaging session. For example, FIG. 1 illustrates an imaging screen 144. During an imaging stage, the workflow module 132 may request or receive the acquired image 140 from the image-processing module, the appropriate graphics from the graphics module 131, and the appropriate information from the database 133 to display. In FIG. 1, the imaging screen 144 includes the ultrasound image 140, the icons 141, and the user-selectable elements 143. The workflow module 132 may determine an overall layout for the display area 117 using the information provided by the other modules.

In an example, the workflow module 132 may select and/or adjust an ultrasound image acquisition protocol, stored in the database 133, for example, and output information via the user interface 122 to guide an operator through the ultrasound image acquisition protocol. The ultrasound image acquisition protocol may include a plurality of anatomical structures to be imaged during an imaging session. Additionally, or alternatively, the ultrasound image acquisition protocol may include a plurality of anatomical features to be measured during the imaging session. The ultrasound image acquisition protocol may only include instructions configured to be output to the user interface, for example, to guide an operator through the acquisition of designated images and/or measurements. However, in other examples, the ultrasound image acquisition protocol may include instructions for the ultrasound system 100 to automatically acquire some or all of the images, fully or partially perform the designated measurements, or other functions.

In one non-limiting example, the ultrasound image acquisition protocol may include a list of fetal diagnostic parameters, such as fetal anatomical structures and fetal anatomical features, to be imaged and/or measured, during a prenatal ultrasound imaging session that may occur during the first and/or second trimester of a pregnancy, for example. The list of fetal diagnostic parameters defined in the ultrasound image acquisition protocol may be predefined, for example by a medical advisory board or other entity, or the diagnostic parameters may be partially or fully dynamically determined, based on operator input or other parameters. Based on the images acquired during the imaging session, as well as measurements taken of various anatomical features of the fetus, fetal growth and development may be monitored. Example anatomical structures that may be imaged include but are not limited to multiple planes and cross-sectional views of the fetal head, heart, abdomen, kidneys, umbilical cord, spine, and limbs, as well as the cervix and placenta. Example anatomical features that may be measured include but are not limited to head circumference, bi-parietal diameter, nuchal fold, nuchal translucency, lateral ventricle diameter, abdominal circumference, renal pelvis diameter, femur length, number of umbilical arteries, and other features. The ultrasound image acquisition protocol may ensure uniformity and accuracy in the image acquisition and feature measurement process, such that each desired anatomical structure and feature is imaged and measured with minor variability between imaging sessions and operators.

As described above, a standard ultrasound image acquisition protocol for monitoring fetal development during pregnancy may include a few soft markers for detecting one or more developmental disorders, such as aneuploidy (e.g., trisomy 21). Such soft markers may include the femur length and nuchal translucency. However, such soft markers are “softly” correlated with a respective developmental disorder, in that the markers for the disorder are also commonly found in fetuses without developmental disorders. Accordingly, accurate diagnosis of a developmental disorder may not be provided on the basis of ultrasound imaging alone. Further, during a standard fetal ultrasound imaging session, due to time and/or cost constraints, only a subset of soft markers for one or more developmental disorders are typically assessed. As such, if a fetus is displaying one or more soft markers for a disorder, follow up testing may be needed, such as additional ultrasound imaging sessions, amniocentesis, chorionic villi sampling, etc. The follow up testing may prolong diagnosis, pose additional risks to the fetus, or otherwise be undesirable.

To provide a more efficient image acquisition and disorder diagnosis process, the workflow module 132 may be configured to adjust the ultrasound image acquisition protocol based on genetic analysis information of the fetus. The genetic analysis information may be indicative of a probability of the fetus having a particular phenotype, such as trisomy 21. As used herein, “phenotype” refers to observable characteristics or traits, such as morphology, development, etc. In one example, the genetic analysis of the fetus is carried out prior to the ultrasound imaging session. The genetic analysis may include a DNA sequence analysis of fetal DNA reconstructed from a maternal blood sample, for example, or other suitable genetic analysis, such as a genetic analysis performed on the parents of the fetus. The genetic analysis information may be stored in a genetic analysis database 154 and automatically accessed by the system controller 115 from a remote device 152 via a network 150, for example. However, in other examples, the genetic analysis information may be stored on the remote device, or stored on the system controller. In a further example, raw (e.g., unprocessed) DNA sequences may be obtained by the system controller 115 from the remote device 152 and/or database 154, and the system controller 115 may process the sequence data to determine a likelihood the fetus is affected by one or more disorders (e.g., determine a probability the fetus will display a given phenotype). In a still further example, the genetic analysis information may be input to the user interface by the operator.

Based on the genetic analysis information, the workflow module 132 may select an appropriate ultrasound image acquisition protocol that provides a targeted list of anatomical structures to be imaged and anatomical features to be measured. This may include making adjustments to a standard ultrasound image acquisition protocol, or it may include selecting a different ultrasound image acquisition protocol tailored for a given phenotype predicted based on the genetic analysis information. In the example of trisomy 21, if the probability that the fetus has trisomy 21 (as determined from the genetic analysis information) is relatively high (e.g., higher than the maternal age-based population risk), the selected image acquisition protocol may include a measurement and/or assessment of a higher number of trisomy 21 soft markers than a standard image acquisition protocol. After the images have been acquired and various features measured according to the selected protocol, one or more comparisons may be made between the measured features and respective population curves. For example, the nuchal translucency measured during the ultrasound imaging session may be compared to a curve of nuchal translucencies for the general population to calculate a percentile that may be one factor used to determine the risk of the fetus having trisomy 21. Based on all the images, measurements, and feature population percentile determinations, the workflow module 132 may be configured to output an updated phenotype probability.

The genetic analysis information may be presented in a suitable form. In one example, as described above, the genetic analysis information may be used to determine the probability the fetus has a give phenotype, such as one or more developmental disorders. In another example, the genetic analysis information may not provide a phenotype probability, but may instead provide a genotype detected by the DNA analysis. This may include a definitive positive or negative for a given disorder. In such cases, assessment of the images acquired during an ultrasound session may provide for a probability that the fetus actually displays any outward characteristics of the disorder (e.g., the phenotype). For example, the results from the DNA analysis may indicate the fetus has a given genotype (at least in some cells of the fetus), but upon analysis of the ultrasound images, it may be determined the fetus does not display the physical characteristics of the disorder, and thus the probability of the phenotype may be lowered after the ultrasound.

Further, it may be difficult to detect from the fetal DNA sequence analysis how severe the disorder will actually present in the fetus, as it is difficult to determine via DNA sequencing if every cell in the fetus includes a particular genetic abnormality. For example, some cases of trisomy 21 may be mosaic, in that only some of the cells of the fetus include extra genetic material from chromosome 21. Mosaic disorders may have variable phenotypic outcomes, with some cases being relatively mild and others being relatively severe. Further, even in cases where every cell has a particular genetic abnormality, additional factors may affect the severity of how the disorder actually presents in the fetus. Thus, in addition to or in alternative of the updated probability, a severity assessment may be provided based on the imaged anatomical structures and measured features. The severity assessment may include comparisons between the measured features and respective population curves, where the population curves are adjusted to reflect only a population for the given phenotype, rather than the general population.

Thus, the ultrasound system described above provides for a system for imaging a fetus including an ultrasound probe to emit ultrasonic signals to a volume of the fetus; an ultrasound receiver to receive echoes of the emitted ultrasonic signals; and a computing system operably connected to the ultrasound probe, ultrasound receiver, and a display device. The computing system includes instructions to obtain genetic analysis information of the fetus and adjust an ultrasound image acquisition protocol based on the information. The computing system may include further instructions to generate a user interface output for display on the display device, where the user interface output includes information to instruct a user to follow the adjusted ultrasound image acquisition protocol.

The ultrasound image acquisition protocol may include a predefined list of anatomical structures and features to be imaged and measured during imaging of the fetus. The computing system may also include instructions to, during adjustment of the ultrasound image acquisition protocol, adjust which anatomical structures are imaged and which anatomical features are measured based on the information. The user interface output may further comprise a comparison of one or more of the measured anatomical features to a population curve. The population curve may be adjusted based on the information.

The computing system may include further instructions to process the received echoes into one or more images for display via the user interface, where the images are acquired according to the adjusted ultrasound image acquisition protocol.

The computing system may obtain raw DNA sequencing data from a remote device and determine a probability of the fetus displaying a particular phenotype based on the raw DNA sequencing data in one example. In another example, the computing system obtains the genetic analysis information from a remote device. In a further example, the computing system obtains the genetic analysis information via user input to the user interface.

The computing system may also include instructions to, during adjustment of the ultrasound image acquisition protocol, adjust a color of one or more images rendered for display on the display device, via the user interface, based on a sex of the fetus, where the sex of the fetus is determined based on the genetic analysis information of the fetus.

Turning now to FIG. 2, a method 200 for imaging a fetus is provided. Method 200 may be performed by a computing system, such as the system controller 115 of the ultrasound system described above with respect to FIG. 1, according to non-transitory instructions stored on and executed by the computing system. At 202, method 200 includes obtaining genetic analysis information of a fetus. The genetic analysis information may be usable to determine a probability that the fetus will display a particular phenotype. The phenotype may be a suitable phenotype that can be predicted via genetic analysis, such as sex of the fetus, or a particular developmental or genetic disorder, such as aneuploidy (e.g., trisomy 13, trisomy 18, trisomy 21, Turner's syndrome, etc.), that effects one or more anatomical structures of the fetus detectable via ultrasound. The probability that the fetus has the given phenotype may be represented in a suitable manner, such as within a range of 0-1, where 0 indicates no risk and 1 represents that the fetus has the phenotype.

The genetic analysis information may be obtained in a suitable manner. In one example, as indicated at 204, the probability of a phenotype may be calculated by the computing system from raw test data. For example, a DNA sequence analysis of fetal DNA may be performed and the sequences stored in a remote device accessible by the computing system of the ultrasound system. The computing system may obtain the DNA sequences and process the sequences to determine if any genetic abnormalities exist, and determine a probability that the fetus has a given phenotype. In another example, the genetic analysis information may be obtained from a remote device, as indicated at 206. In a further example, indicated at 208, the information may be obtained via user input, to a user interface of the computing system, for example.

At 210, method 200 includes selecting an ultrasound image acquisition protocol based on the information. This may include, at 212, selecting an image acquisition protocol for a particular phenotype if the probability of the fetus having that phenotype, as determined by the genetic analysis of the fetus, is greater than a threshold. If the probability is less than the threshold, the standard image acquisition protocol may be selected, as indicated at 214, to avoid the collection of unnecessary images, for example. In this way, a particular ultrasound image acquisition protocol may be automatically selected by the computing system based on the probability the fetus has a given phenotype, in order to tailor the imaging session to the individual fetus, rather than performing the same, standard protocol on all fetuses.

The various ultrasound image acquisition protocols may differ in one or more manners, such as different number of acquired images, different order of acquired images, different anatomical structures imaged, different anatomical features measured, etc. For example, a first ultrasound image acquisition protocol may include acquiring a first number of images, while a second ultrasound image acquisition protocol may include acquiring a second, different number of images, where the images acquired according to the first protocol do not overlap with the images acquired according to the second protocol, or where the images from the first protocol partially overlap with the images from the second protocol.

In another example, the first ultrasound image protocol may include a set of images acquired in a first order, and the second ultrasound image protocol may include a set of images acquired in a second order. The images in the first and second set may fully or partially overlap. This may include, in an example, the first ultrasound image acquisition protocol having instructions to acquire a first image (e.g., image A), then acquire a second image (e.g., image B), and then acquire a third image (e.g., image C). The second ultrasound image acquisition protocol may include instructions to acquire the second image (image B), then acquire the first image (image A), and then acquire a fourth image, not included in the images of the first image acquisition protocol (e.g., image D).

In a further example, the ultrasound image acquisition protocols may differ in the ultrasound parameters used to acquire the images. This may include adjusting one or more of ultrasound signal transmission parameters, ultrasound signal receive parameters, ultrasound signal processing parameters, or ultrasound signal display parameters.

At 216, instructions are output for display on a user interface in order to guide an operator through the selected protocol. The instructions may include which anatomical features to image, in which order, and may also include which measurements of which anatomical features should be taken. Further, at 218, the method includes acquiring images and/or performing measurements following the selected protocol. The images may be acquired via operator input or automatically by the ultrasound system. Likewise, the measurements may be taken via operator input, automatically by the ultrasound system, or both.

At 220, method 200 includes calculating a fetal characteristic using a respective population curve, where the population curve is selected based on the information. The fetal characteristic may include a percentile for a given anatomical feature, such as femur length. Typically, during standard fetal ultrasound imaging sessions, the femur length of the imaged fetus is compared to a general population femur length curve in order to determine the percentile femur length of the imaged fetus. However, based on the genetic analysis information, the population curve may be adjusted to include only femur lengths for a population of fetuses affected by a particular phenotype indicated by the genetic analysis information. This may assist in predicting the severity of the phenotype, for example.

At 222, an updated phenotype probability and/or a determined severity of the phenotype is output for display via the user interface, based on an analysis of the acquired images (e.g., based on the measurements of certain anatomical features and comparisons to respective population curves). In this way, a phenotype predicted based on a genotype detected by the genetic analysis may be adjusted according to the actual physical traits of the fetus as imaged during the ultrasound imaging session.

Additionally, in some embodiments, the genetic analysis information obtained at 202 may include a sex of the fetus. Accordingly, at 224, method 200 may optionally include adjusting a color of one or more rendered images of the fetus displayed via the user interface based on the information. For example, if the fetus is a girl, the images may be rendered in pink, while if the fetus is a boy, the images may be rendered in blue.

Thus, an embodiment of a method for imaging a fetus comprises obtaining genetic analysis information of the fetus; and adjusting an ultrasound image acquisition protocol based on the information. The ultrasound image acquisition protocol may comprise a list of diagnostic parameters to be obtained during ultrasound imaging of the fetus. The diagnostic parameters may comprise one or more of imaged anatomical structures and measured anatomical features. The ultrasound image acquisition protocol may be adjusted based on the information to adjust which anatomical structures of the fetus are imaged. Further, adjusting the ultrasound image acquisition protocol based on the information may comprise adjusting which anatomical features of the fetus are measured based on the probability.

The ultrasound image acquisition protocol may further comprise a determination of a characteristic of the fetus, such as a percentile of a measurement of an anatomical feature, based on a comparison of one or more measured anatomical features of the fetus to a population curve. Adjusting the ultrasound image acquisition protocol based on the information may further comprise adjusting the population curve based on the information.

The genetic analysis information may include a probability of the fetus having a given genetic disorder, and the ultrasound image acquisition protocol may be adjusted based on the probability of the fetus having the given genetic disorder. This may include, if the probability is above a threshold, adjusting the ultrasound image acquisition protocol to include a first set of diagnostic parameters; and if the probability is below the threshold, adjusting the ultrasound image acquisition protocol to include a second set of diagnostic parameters, different than the first set. It is to be understood that the first and second set of diagnostic parameters may include at least some overlapping diagnostic parameters. Further, the first and second sets of diagnostic parameters may differ in one or more of number of images acquired, order of images acquired, anatomical structures imaged, planes and/or views of anatomical structures acquired, and anatomical features measured.

In an example, the ultrasound image acquisition protocol may be adjusted to adjust an order in which a plurality of images of the fetus are taken. This may include, for a first ultrasound image acquisition protocol, acquiring the plurality of images according to a first order, the first order including first acquiring a first image, then acquiring a second image, and then acquiring a third image. For a second ultrasound image acquisition protocol, the plurality of images may be acquired according to a second image order, the second image order including first acquiring the second image, then acquiring the first image, and then acquiring the third image. In some examples, the second image order may further include acquiring a fourth image, where the fourth image is not acquired during the first image acquisition protocol.

The method may further comprise obtaining a plurality of images of anatomical structures of the fetus and a plurality of measurements of anatomical features of the fetus following the adjusted ultrasound image acquisition protocol, and generating one or more of an updated probability of the phenotype and a severity assessment of the phenotype based on the plurality of images and the plurality of measurements.

The genetic analysis information may be obtained from a remote server in one example. The genetic analysis may include a DNA analysis of the fetus performed on fetal DNA reconstructed from a maternal blood sample. In one example, the genetic analysis information may be usable to determine a probability the fetus has a given phenotype, such as a genetic disorder. Example genetic disorders include trisomy 13, trisomy 18, and trisomy 21. In further examples, the genetic disorder may comprise virtually any genetic abnormality, including but not limited to genetic abnormalities that affect the development of a fetus. The genetic analysis information may be indicative of a sex of the fetus in another example. The ultrasound image acquisition protocol may be adjusted based on the sex of the fetus, to include adjusting a color of one or more rendered images of the fetus during imaging of the fetus based on the sex of the fetus.

In another example, the ultrasound image acquisition protocol may be adjusted based on the genetic information by adjusting one or more of ultrasound signal transmission parameters, ultrasound signal receive parameters, ultrasound signal processing parameters, or ultrasound signal display parameters.

While the description provided above discloses a method applicable for selecting an ultrasound image acquisition protocol based on virtually any phenotype that may be displayed by a fetus, in practice some developmental disorders are more common than others and/or may present with more soft markers detectable via ultrasound. One such disorder is trisomy 21 (also known as Down syndrome). FIG. 3 illustrates a method 300 for selecting an ultrasound image acquisition protocol based on the probability a fetus has trisomy 21.

At 302, method 300 includes obtaining a probability that a fetus has trisomy 21. The probability may be determined from genetic analysis information obtained according to the embodiments described above with respect to FIGS. 1 and 2. At 304, method 300 determines if the probability is greater than a threshold. The threshold may be a suitable threshold that indicates trisomy 21 is likely, including greater than zero, greater than the average population risk for trisomy 21, or other suitable threshold. In some examples, the probability may be a definitive genotype, where it is indicated that the fetus either has trisomy 21 or does not have trisomy 21. In such cases, the fetus having trisomy 21 would be considered a probability greater than the threshold.

If the probability is greater than the threshold, method 300 proceeds to 306 to select a trisomy 21 image acquisition protocol. At 308, method 300 includes acquiring images of anatomical structures and/or taking measurements of anatomical features according to the selected trisomy 21 protocol. This includes, as indicated at 310, measuring a first, greater number of trisomy 21 soft markers. The soft markers may include one or more of nuchal translucency, femur length, choroid plexus cysts, intracardiac echogenic foci, echogenic bowel, umbilical artery state, renal pelvis state, and other soft markers.

At 312, one or more soft marker measurements are compared to a respective trisomy 21 population curve. At 314, an updated probability of the fetus having trisomy 21 is output based on the soft marker measurements and comparisons. At 316, a severity of trisomy 21 predicted for the fetus is output, also based on the soft marker measurements and comparisons.

Thus, if a genetic analysis performed on the fetus indicates it is likely the fetus has trisomy 21, an ultrasound image acquisition protocol specific for trisomy 21 is followed. The trisomy 21 image acquisition protocol may guide an operator of the ultrasound system to acquire a different set of images of the fetus than a standard fetal ultrasound imaging session, and/or it may guide the operator to measure or assess a different set of soft markers, in particular it may guide the operator to measure more defined soft markers for trisomy 21. In doing so, the probability the fetus will eventually present with trisomy 21 may be adjusted based on the anatomy of the fetus as determined by the ultrasound, and/or the severity of trisomy 21 affecting the fetus may be determined.

In contrast, returning to 304, if the probability that the fetus has trisomy 21 is less than the threshold, method 300 proceeds to 320 to select the standard image acquisition protocol, and at 322, acquire images of anatomical structures and/or take measurements of anatomical features according to the standard protocol. This includes, as indicated at 324, measuring a second, fewer number of trisomy 21 soft markers.

At 326, the one or more soft markers are compared to respective general population curves, and at 328, an updated probability of the fetus having trisomy 21 is output, based on the soft marker measurements and comparisons.

FIG. 4 illustrates example user interface outputs that may be displayed during two different exemplary ultrasound image acquisition protocols. A standard ultrasound image acquisition protocol 400 is illustrated across a top row of FIG. 4, and an adjusted ultrasound image protocol 420 for trisomy 21 is illustrated across a bottom row of FIG. 4. As described above, the standard ultrasound image acquisition protocol 400 may be performed on a fetus whose genetic analysis information indicates the fetus is not at elevated risk for trisomy 21, while the adjusted ultrasound image acquisition protocol 420 may be performed on a fetus whose genetic analysis information indicates the fetus has trisomy 21 or is at elevated risk for trisomy 21.

During an ultrasound session where images are acquired according to the standard ultrasound image acquisition protocol 400, a number of anatomical structures are imaged, and a number of anatomical features are measured, in order to monitor the growth and development of the fetus, as well as screen for possible developmental disorders. Three example user interface outputs that may displayed during the standard image acquisition protocol 400 are illustrated, a first user interface output 402, a second user interface output 410, and a third user interface output 414. The first user interface output 402 includes an acquired image of a head 404 of a fetus. The image of the head 404 may include various structures, such as the cerebellum and other features, as well as the nuchal fold. A measurement of the nuchal fold 406 may be taken, and compared to a general population curve 408 of nuchal fold sizes for age-matched fetuses. Based on the comparison, a percentile nuchal fold size for the fetus may be determined. In the example presented in FIG. 4, the fetus has a below-average nuchal fold size (e.g., in the range of the 25^{th}-30^{th} percentile), which may indicate, for the nuchal fold soft marker, a relatively low risk of the fetus having trisomy 21 or other developmental disorder.

The standard ultrasound image acquisition protocol 400 may include a plurality of other images of the head, as well as a plurality of images of other anatomical structures. The second user interface output 410 illustrates an image of the heart 412 of the fetus, and the third user interface output 414 illustrates an image of the abdomen 416 of the fetus. The user interface outputs are provided as illustration only and are not meant to be limiting, as other user interface outputs, including other images of other structures, are possible.

During an ultrasound session where images are acquired according to the adjusted standard ultrasound image acquisition protocol 420 for trisomy 21, a number of anatomical structures are imaged, and a number of anatomical features are measured, in order to monitor the growth and development of the fetus, as well determine the severity of trisomy 21. Three example user interface outputs that may be displayed during the adjusted image acquisition protocol 420 are illustrated, a first user interface output 422, a second user interface output 430, and a third user interface output 434. The first user interface output 422 includes an acquired image of a head 424 of a fetus, similar to the image of the head 404 acquired during the standard ultrasound image acquisition protocol 400. A measurement of the nuchal fold 426 may be taken, and compared to an adjusted population curve 428 of nuchal fold sizes for age-matched fetuses known to have trisomy 21. Based on the comparison, a percentile nuchal fold size for the fetus, as a percentile of a trisomy 21 population, may be determined. In the example presented in FIG. 4, the fetus has an above-average nuchal fold size for the general population (if it were plotted on the population curve 408), as would be expected for a fetus with trisomy 21. However, the fetus has an average nuchal fold size for fetuses with trisomy 21 (e.g., in the range of the 50^{th} percentile), which may indicate, at least for the nuchal fold soft marker, that the fetus has trisomy 21 with average severity.

The adjusted ultrasound image acquisition protocol 420 for trisomy 21 may include a plurality of other images of the head, as well as a plurality of images of other anatomical structures. At least some of these images may differ from the images of the standard ultrasound image acquisition protocol 400. The second user interface output 430 illustrates an image of the heart 432 of the fetus, similar to the image of the heart 412 from the standard ultrasound image acquisition protocol 400. However, the adjusted ultrasound image acquisition protocol 420 may include more images of the heart than the standard protocol. This is because heart defects are a known complication of trisomy 21, and thus it may be desirable to image and assess the heart of a fetus with trisomy 21 in more detail than a fetus without trisomy 21. Accordingly, the third user interface output 434 illustrates another image of the heart 436 of the fetus, not acquired during the standard protocol illustrated in FIG. 4. While not shown in FIG. 4, an image of an abdomen may also be acquired during the adjusted ultrasound image acquisition protocol 420, albeit later in the acquisition protocol. Similar to above, the user interface outputs are provided as illustration only and are not meant to be limiting, as other user interface outputs, including other images of other structures, are possible.

While the above described methods disclose how an ultrasound protocol may be adjusted based on the probability of a fetus having one particular genetic abnormality, it should be understood that the ultrasound system may be provided with more than one probability, for more than one genetic abnormality. As such, the ultrasound system may know that the fetus has a low risk for all other tested abnormalities, and thus the ultrasound protocol may be focused on only trisomy 21. However, it may be possible for the fetus to have elevated risk for multiple genetic disorders. In such cases, multiple ultrasound protocols may be followed, or multiple protocols may be combined and performed together.

Thus, the methods and systems described above provide for a system for imaging a fetus. In an example, the system comprises an ultrasound probe to emit ultrasonic signals to a volume of the fetus; an ultrasound receiver to receive echoes of the emitted ultrasonic signals; and a computing system operably connected to the ultrasound probe, ultrasound receiver, and a display device. The computing system includes instructions to: obtain information indicative of a probability of the fetus having a given genetic disorder based on a DNA sequence analysis of the fetus; select an ultrasound image acquisition protocol based on the information, the ultrasound image acquisition protocol comprising a predefined list of soft markers of the given genetic disorder to be measured; acquire a plurality of images of the fetus according to the selected ultrasound image acquisition protocol, each image processed by the computing system from the echoes received by the ultrasound receiver; measure the soft markers from the acquired images; compare one or more of the measured soft markers to a respective population curve; and determine an updated probability of the genetic disorder and/or perform a severity assessment of the genetic disorder based on the comparison.

In one example, the genetic disorder is trisomy 21. If the probability of the fetus having trisomy 21 is greater than a threshold, the selected ultrasound image acquisition protocol may have a first, larger number of trisomy 21 soft markers to be measured, and if the probability the of the fetus having trisomy 21 is less than the threshold, the selected ultrasound image acquisition protocol may have a second, smaller number of trisomy 21 soft markers to be imaged. The trisomy 21 soft markers may include one or more of nuchal translucency, femur length, choroid plexus cysts, intracardiac echogenic foci, echogenic bowel, umbilical artery state, and renal pelvis state.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal language of the claims.

1. A method for imaging a fetus, comprising:
obtaining genetic analysis information of the fetus; and
adjusting an ultrasound image acquisition protocol based on the information.

2. The method of claim 1, wherein the ultrasound image acquisition protocol comprises a list of diagnostic parameters to be obtained during ultrasound imaging of the fetus, the diagnostic parameters comprising one or more of imaged anatomical structures and measured anatomical features.

3. The method of claim 2, wherein adjusting the ultrasound image acquisition protocol based on the information comprises adjusting which anatomical structures of the fetus are imaged based on the information.

4. The method of claim 2, wherein adjusting the ultrasound image acquisition protocol based on the information comprises adjusting which anatomical features of the fetus are measured based on the information.

5. The method of claim 2, wherein the ultrasound image acquisition protocol further comprises a determination of a characteristic of the fetus based on a comparison of one or more measured anatomical features of the fetus to a population curve, and wherein adjusting the ultrasound image acquisition protocol based on the information further comprises adjusting the population curve based on the information.

6. The method of claim 2, wherein the genetic analysis information comprises a probability of the fetus having a given genetic disorder, and wherein adjusting the ultrasound image acquisition protocol based on the information comprises:
if the probability is above a threshold, adjusting the ultrasound image acquisition protocol to include a first set of diagnostic parameters; and
if the probability is below the threshold, adjusting the ultrasound image acquisition protocol to include a second set of diagnostic parameters, different than the first set.

7. The method of claim 6, further comprising obtaining a plurality of images of anatomical structures of the fetus and a plurality of measurements of anatomical features of the fetus following the adjusted ultrasound image acquisition protocol, and generating one or more of an updated probability of the fetus having the genetic disorder and a severity assessment of the genetic disorder based on the plurality of images and the plurality of measurements.

8. The method of claim 1, wherein obtaining the genetic analysis information comprises obtaining the information from a remote server, and wherein the genetic analysis comprises a DNA analysis of the fetus performed on fetal DNA reconstructed from a maternal blood sample.

9. The method of claim 1, wherein the genetic analysis information is indicative of a sex of the fetus, and wherein adjusting the ultrasound image acquisition protocol based on the information comprises adjusting a color of one or more rendered images of the fetus during imaging of the fetus based on the sex of the fetus.

10. The method of claim 1, wherein adjusting the ultrasound image acquisition protocol includes adjusting an order in which a plurality of images of the fetus are taken.

11. The method of claim 1, wherein adjusting the ultrasound image acquisition protocol based on the genetic information comprises adjusting one or more of ultrasound signal transmission parameters, ultrasound signal receive parameters, ultrasound signal processing parameters, or ultrasound signal display parameters.

12. A system for imaging a fetus, comprising:
an ultrasound probe to emit ultrasonic signals to a volume of the fetus;
an ultrasound receiver to receive echoes of the emitted ultrasonic signals; and
a computing system operably connected to the ultrasound probe, ultrasound receiver, and a display device, the computing system including instructions to:
obtain genetic analysis information of the fetus;
adjust an ultrasound image acquisition protocol based on the information; and
generate a user interface for display on the display device, the user interface comprising information to instruct a user to follow the adjusted ultrasound image acquisition protocol.

13. The system of claim 12, wherein the ultrasound image acquisition protocol comprises a predefined list of anatomical structures and features to be imaged and measured during imaging of the fetus, and wherein the computing system includes instructions to, during adjustment of the ultrasound image acquisition protocol, adjust which anatomical structures are imaged and which anatomical features are measured based on the information.

14. The system of claim 13, wherein the user interface further comprises a comparison of one or more of the measured anatomical features to a population curve, and wherein the population curve is adjusted based on the information.

15. The system of claim 12, wherein the computing system includes further instructions to process the received echoes into one or more images for display via the user interface, the images acquired according to the adjusted ultrasound image acquisition protocol.

16. The system of claim 12, wherein the computing system obtains raw DNA sequencing data from a remote device and determines a probability of the fetus displaying a particular phenotype based on the raw DNA sequencing data.

17. The system of claim 12, wherein the computing system obtains the information from a remote device.

18. The system of claim 12, wherein the genetic analysis information is indicative of a sex of the fetus, and wherein the computing system includes instructions to, during adjustment of the ultrasound image acquisition protocol, adjust a color of one or more rendered images of the fetus during imaging of the fetus based on the sex of the fetus.

19. A system for imaging a fetus, comprising:
an ultrasound probe to emit ultrasonic signals to a volume of the fetus;
an ultrasound receiver to receive echoes of the emitted ultrasonic signals; and
a computing system operably connected to the ultrasound probe, ultrasound receiver, and a display device, the computing system including instructions to:
obtain information indicative of a probability of the fetus having a given genetic disorder based on a DNA sequence analysis of the fetus;
select an ultrasound image acquisition protocol based on the information, the ultrasound image acquisition protocol comprising a predefined list of soft markers of the given genetic disorder to be measured;
acquire a plurality of images of the fetus according to the selected ultrasound image acquisition protocol, each image processed by the computing system from the echoes received by the ultrasound receiver;
measure the soft markers from the acquired images;
compare one or more of the measured soft markers to a respective population curve; and
determine an updated probability of the fetus having the genetic disorder and/or perform a severity assessment of the genetic disorder based on the comparison.

20. The system of claim 19, wherein the genetic disorder is trisomy 21, and wherein:
if the probability of the fetus having trisomy 21 is greater than a threshold, the selected ultrasound image acquisition protocol has a first, larger number of trisomy 21 soft markers to be measured, and
if probability the of the fetus having trisomy 21 is less than the threshold, the selected ultrasound image acquisition protocol has a second, smaller number of trisomy 21 soft markers to be imaged.
